(19) United States
(12) Patent Application Publication
(10) Pub. No.: US 2018/0289052 A1
(43) Pub. Date: Oct. 11, 2018

(54) PROBIOTIC FORMULATION

(71) Applicant: Bifodan A/S, Hundested (DK)

(72) Inventors: Kasper Hallestad Wennike, Værløse (DK); Suzanne Ali Yassine, Copenhagen NV (DK)

(21) Appl. No.: 15/766,558

(22) PCT Filed: Oct. 7, 2016
(86) PCT No.: PCT/EP2016/074081

§ 371 Date: Apr. 6, 2018

(30) Foreign Application Priority Data
Oct. 7, 2015 (EP) 15188842.7

(51) Int. Cl. A23L 29/30; A23L 33/135; A61K 35/745; A61K 35/747

(52) U.S. Cl. CPC A23L 29/37; A23L 33/135; A61K 35/745; A61K 35/747; A23V 2002/00; A23V 2200/02; A23Y 2300/49; A23V 2250/032; A23V 2250/161; A23V 2250/1628; A23V 2250/6402; A23V 2250/6422; A23Y 2220/73; A23V 2200/3204

(57) ABSTRACT

A nutritional composition comprising at least one microbial organism formulated as a fast melting composition.

### FIELD OF THE INVENTION

The present invention relates to the field of nutrition. In particular, the present invention relates to the field of oral nutritional supplements.

### BACKGROUND OF THE INVENTION

Probiotics are live microorganisms or microbial mixtures administered to improve the patient's microbial balance, particularly the environment of the gastrointestinal tract. The presence of e.g. Lactobacilli is important for the maintenance of the intestinal microbial ecosystem. Lactobacilli have been shown to possess inhibitory activity toward the growth of pathogenic bacteria such as Listeria monocytogenes, Escherichia coli, Salmonella spp and others. This inhibition could be due to the production of inhibitory compounds such as organic acids, hydrogen peroxide, bacteriocins or reuterin or to competitive adhesion to the epithelium.

A variety of compositions for supplementing probiotics are currently available. The compositions are typically provided for improving the microflora within a gastrointestinal (GI) tract of subjects. Current formulation technologies include the utilization of encapsulation and stabilization techniques for shielding the probiotics with a protective layer such that the composition comprising the microorganism may be delivered to GI tract of the subject. Further, the focus of many formulation technologies has been to protect the viability of probiotics during distribution and storage.

However, there is a remaining need for formulation of probiotics having properties that will associate the ingestion with a pleasant taste and mouthfeel.

### SUMMARY OF THE INVENTION

The present invention was made in view of the prior art described above, and the object of the present invention is to provide a probiotic formulation having improved taste and mouthfeel.

To solve the problem, the present invention a nutritional composition as described in the below first aspect.

A first aspect of the present invention concerns a nutritional composition comprising:
(i) at least one live microbial organism,
(ii) at least one low calorie sweetener,
(iii) at least one hygroscopic agent,
(iv) at least one aroma compound,

The composition may optionally comprise one or more acidity regulator and/or one or more antioxidant.

A second aspect of the present invention concerns a container containing the composition of the present invention. A third aspect provides a kit comprising a plurality of said containers of the present invention.

A fourth aspect of the present invention provides a method for preparing a composition containing a microbial organism, said method comprising the steps of:
(a) mixing at least one microbial organism with at least one low calorie sweetener, at least one hygroscopic agent, at least one aroma compound, and optionally at least one acidity regulator and/or an antioxidant agent,
(b) reducing the water activity in the mixture obtained under step (a) to be less than 15%, such as less than 10%, such as in the range of 5% to 10% water activity,
(c) packaging the mixture obtained under step (b) in a container.

The composition obtainable from the above method is provided in a further aspect.

Yet a further aspects of the present invention concerns method for and use of the composition of the present invention for stimulating mucin production, stimulating of the stabilization of intestinal barrier, providing antimicrobial substances to the gut, stimulating gut transit, and/or stimulating the immune system of a subject.

### BRIEF DESCRIPTION OF THE DRAWINGS

FIGS. 1 to 3 shows the particle size distribution in a sample preparation of a composition of the present invention.

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

Definitions

Microbial Organism

In the context of the present invention the term “live microbial organism” refers to a microorganism that when ingested in adequate amounts by a subject (such as in the form a formulation as described herein) confer a health benefit to the subject. A probiotic microorganism is a live microorganism which, when administered in adequate amounts, confer a health benefit to the host by influencing the composition and or metabolic activity of the flora of the gastrointestinal (GI) tract (FAO/WHO 2001). Health benefit reported includes (i) improved digestion of lactose and reduce intestinal bloating, flatulence and discomfort; (ii) prevention of traveller's diarrhoea; (iii) enhancing the immune system, improving resistance to infection and improving well-being; (iv) lowering serum cholesterol levels and reducing the incidence of coronary heart disease; (v) treating intractable diarrhoea following antibiotic therapy; (vi) reducing allergic inflammation.

Low Calorie Sweetener

Low calorie sweeteners refer to substances added to foods, drinks and nutritional supplements to provide sweet taste without calories, or at least with very few calories. Most low calorie sweeteners are several hundred times sweeter than table sugar, meaning that only small quantities need to be added to achieve a sweetening effect. These low calorie sweeteners are also referred to as high-intensity sweeteners (or intense sweeteners). Non-limiting examples of intense sweeteners include alitame, acesulfame potassium (Ace K), aspartame, advantame, cyclamate, neotame, saccharin, sucralose, steviol glycosides and thaumatin.

Some non-sugar sweeteners are polyols, also known as “sugar alcohols”. These are, in general, less sweet than sucrose but have similar bulk properties and can be used in a wide range of food products. Sometimes the sweetness profile is ‘fine-tuned’ by mixing with high-intensity sweeteners.

Useful sugar alcohols include erythritol and xylitol, which are typically used to sweeten chewing gum, candy, fruit spreads, toothpaste, cough syrup, and other products.

Hygroscopic Agent

The term hygroscopic agent (also referred to as moisture scavenger) refers to an additive used to chemically lock-up water in the composition in order to control the water activity in the formulation.

Aroma Compound

In the context of the present invention an aroma compound, also referred to as aroma, is a chemical compound that has a smell or odor. A chemical compound has a smell or odor when it is sufficiently volatile to be transported to the olfactory system in the upper part of the nose. Generally molecules meeting this specification have molecular weights of <300. One group of aroma compounds, flavors, affect both the sense of taste and smell. Flavors are typically naturally occurring. Non-limiting examples of aroma compounds useful in context of the present invention includes honeydew melon aroma, blueberry aroma, peach aroma, strawberry aroma, raspberry aroma, cola aroma, chocolate aroma, peppermint aroma, cherry aroma, lemon aroma, lime aroma, orange aroma, vanilla aroma, tangerine aroma, liquorice aroma, apricot aroma, eucalyptus aroma, green tea aroma, ginger aroma and bilberry aroma.

Acidic Regulators

In the context of the presence invention acidic regulators may be included to regulate and control the pH of the composition. The acidic regulator may also be contributing to the flavour of the composition. Acidic regulators include, but are not limited to, citric acid, acetic acid, calcium acetate, lactic acid, malic acid, fumaric acid and tartaric acid.

Antioxidant

The context of the present invention the term “antioxidant” refers mainly to non-nutrient compounds, which have antioxidant capacity in vitro. Dietary antioxidant vitamins include vitamin A, vitamin C and vitamin E. Preferred antioxidants include but are not limited to sodium ascorbate and alpha tocopherol.

Water Activity

Water activity is the ratio of the vapor pressure of water in a material (p) to the vapor pressure of pure water (po) at the same temperature. Relative humidity of air is the ratio of the vapor pressure of air to its saturation vapor pressure. When vapor and temperature equilibrium are obtained, the water activity of the sample is equal to the relative humidity of air surrounding the sample in a sealed measurement chamber. Multiplication of water activity by 100 gives the equilibrium relative humidity (ERH) in percent.

aw=p/po=ERH (%)/100

As described by the above equation, water activity is a ratio of vapor pressures and thus has no units. It ranges from 0.0 aw (bone dry) to 1.0 aw (pure water).

Nutritional Composition of the Present Invention

A first aspect of the present invention concerns a nutritional composition comprising:
(i) at least one live microbial organism,
(ii) at least one low calorie sweetener,
(iii) at least one hygroscopic agent,
(iv) at least one aroma compound,
(v) optionally, at least one acidity regulator, and
(vi) optionally, at least one antioxidant.

Most preferably, the composition is in the form of a powder. In one embodiment, the average particle size of the powder is not larger than 200 micrometer (μm). In another embodiment, the average particle size of the powder is in the range of 45 to 200 micrometer (μm), such as 60 to 125 micrometer (μm), such as 60 to 100 micrometer, for example 75 to 90 micrometer, for example 80 micrometer. In a preferred embodiment, the average particle size of the powder is in the range of 75 to 90 micrometer (μm), for example 80 micrometer.

In one embodiment, the 35 to 95% of the particles have a particle size not larger than 200 micrometer (μm), for example at 50 to 90% of the particles have a size not larger than 200 micrometer (μm), such as at 60 to 80% of the particles have a size not larger than 200 micrometer (μm)

In one embodiment, at least 85% of the particles have a size, which falls within the range of 32 to 500 micrometer, for example at least 88% of the particles have a size in the range of 32 to 500 micrometer (μm).

The inventors have found that the particles size distribution contributes to the pleasant experience upon ingestion. i.e. the fast melting mouthfeel.

The composition of the present invention comprises at least one live microbial organism. The microbial organism (preferably a bacterium) confers a health benefit to the subject, when ingested in adequate amounts by a subject (such as in the form a formulation as described herein). It follows that the microbial organism is non-pathogenic and does not confer any harmful effect in the ingested amounts. The microbial organism is live, which may be confirmed by plating the microbial organism on a suitable medium (e.g. solidified agar in a standard sized Petri dish) and determine the number of colonies formed. The measure, colony forming unit (or CFU), is used quantify the amount of viable (live) microorganism in the composition (reflecting the capacity of the microorganism to replicate).

The initial colony forming units (CFU) and the continued stability and viability of the composition is influenced by a variety of factors. The stability of a probiotic composition tested at the time of manufacture will depend on a combination of factors. Variations in packaging, temperature, and humidity will affect the viability of probiotic products before they are taken. Protective factors that help to preserve the freshness and viability of the probiotic strains in a supplement include refrigeration, resistant packaging, and storage in a cool, dry place. The CFU in the composition declines, if a probiotic composition is held in conditions that are very warm or moist. Thus, the continued stability and viability is dependent on limiting their exposure to stimulating environmental conditions such as warmth and moisture.

Thus, the initial colony forming units (CFU) and the continued stability and viability of the composition partly depend on the amount of moist in the composition. As described herein, the composition is packaged and store in containers (preferably sealed container) in order to provide oxygen and moisture barrier in order to maintain the integrity of the composition. Further, prior packaged the composition the amount of moist in the composition is reduced by subjecting the composition to a step of drying. The inventors have found that higher initial colony forming units (CFU) is obtained if the composition is subjected to a drying step rather than subjecting the individual ingredients to a drying step prior to mixing the ingredients to form the composition.

The water activity of the composition is therefore reduced before the composition is packaged in containers and the containers are sealed. The water activity in the composition is typically less than 0.15 and preferably less than 0.10, such as in the range of 0.05 to 0.10 water activity. The water activities referred to in the context of the compositions of the present are the initial water activities of the composition, i.e. the water activity of the composition immediately after the preparation of the same, e.g. the water activity of the composition immediately after is has been transferred to a container and sealed.

The colony forming units (CFU) referred to in the context of the composition of the present invention are CFU in a single dose and the CFU of the composition shortly after the preparation of the composition.

In one embodiment of the present invention, the colony forming units (CFU) of said microorganism in one dose of the composition is in the range of 10e3 (1000) to 10e12 (1000000000000), such as such as 10e6 to 10e12 colony forming units. In a preferred embodiment, one dose of the composition comprises 10e7 (10000000) to 10e11 (100000000000) colony forming units, such as 10e7 to 10e10 (10000000000) colony forming units, for example 10e7 (10000000) to 10e11 (100000000000) colony forming units, such as 10e7 to 10e10 colony forming units of a probiotic bacterium. In one embodiment, the at least one microbial organism is present in an amount from 0.5 to 5% w/w in said composition.

Preferably the composition is packaged in suitable containers, such as sealed aluminium foil sticks, where each stick comprises one dose of the composition, i.e. one dose of the microorganism. Unless individually packaged as preferred, the container comprising the composition will typically have a recommended serving size listed and then the colony forming units in that serving size.

The live microbial organism used in the context of the present invention may be any live microbial organism, which confers a health benefit to the subject, when ingested in adequate amounts by the subject. In one embodiment, microbial organism is a probiotic fungus. In a preferred embodiment, the microbial organism is a probiotic bacterium. The composition may comprises a single species or strain of microbial organism, or it may comprise a combination of more species or strains of microbial organisms, for examples two or more strains of a probiotic bacterium.

In one embodiment of the present invention, the at least one microbial organism is a probiotic gram positive bacterium. In a further embodiment, the at least one microbial organism is a Lactobacillales. In yet a further embodiment, the Lactobacillales is selected from the list consisting of a Lactobacillus spp., Leuconostoc spp., Pediococcus spp., Lactococcus spp., Streptococcus spp., Aerococcus spp., Carnobacterium spp., Enterococcus spp., Oenococcus spp., Sporolactobacillus spp., Tetragenococcus spp., Vagococcus spp., and Weisella spp. In a preferred embodiment, the Lactobacillales is a Lactobacillus spp. selected from the group consisting of Lactobacillus rhamnosus, Lactobacillus paracasei, Lactobacillus reuteri Lactobacillus acidophilus, Lactobacillus helveticus, Lactobacillus casei, Lactobacillus salivarius, Lactobacillus plantarum, Lactobacillus fermentum, Lactobacillus johnsonii, and Lactobacillus gasseri. In an even more preferred embodiment, the at least one microbial organism is a Lactobacillus strain selected from the group consisting of Lactobacillus rhamnosus GG (ATCC 53103), Lactobacillus rhamnosus SP1 (DSM 21690), Lactobacillus rhamnosus CGMCC 1.3724, Lactobacillus reuteri (ATCC 55730), Lactobacillus reuteri (DSM 17938) and Lactobacillus johnsonii (NCC533; CNCM 1-1225).

In one embodiment of the present invention, the at least one microbial organism is a Lactococcus ssp. such as a Lactococcus ssp. selected from the group consisting of Lactococcus lactis, Lactococcus cremoris, Lactococcus diacetylactis.

In another embodiment of the present invention, the at least one microbial organism is a Bifidobacteriales. In a further embodiment, the at least one microbial organism is a Bifidobacterium spp. such as a Bifidobacterium spp. selected from the group consisting of Bifidobacterium lactis, Bifidobacterium longum, Bifidobacterium breve, Bifidobacterium infantis, Bifidobacterium animalis, Bifidobacterium bifidum and Bifidobacterium adolescentis. In an preferred embodiment, the at least one microbial organism is a Bifidobacterium strain selected from the group consisting of Bifidobacterium Lactis BI-04, Bifidobacterium lactis CNCM 1-3446 (Bb12), Bifidobacterium longum NCC3001, ATCC BAA-999 (BB536), Bifidobacterium breve Bb-03, Bifidobacterium breve M-16V, Bifidobacterium breve R0070 and Bifidobacterium infantis.

In order to obtain the desired health benefit to the subject, it may be advantageous to include more than one microorganism in the composition. Thus, the composition may comprise more than one species/strain of microorganisms, such as two, three, four, five or a higher plurality of species/strains of microorganisms. In one embodiment, the composition comprises at least two species of probiotic bacteria, for example at least one Lactobacillus spp and at least one Bifidobacterium spp., for example Bifidobacterium Lactis BI-04 and one Lactobacillus spp. or Lactobacillus Rhamnosus GG and one Bifidobacterium spp. In a preferred embodiment, the composition comprises Lactobacillus Rhamnosus GG and Bifidobacterium Lactis BI-04, preferably Lactobacillus Rhamnosus GG and Bifidobacterium Lactis BI-04 and no further microorganisms

The composition of the present invention further comprises at least one low calorie sweetener. In one embodiment, the at least one low calorie sweetener selected from the list consisting of a bulk sweetener and an intense sweetener. In yet an embodiment, the low calorie sweetener is a sugar alcohol. In a further embodiment, the low calorie sweetener is selected from the list consisting of xylitol, sorbitol, erythritol, maltitol, lactitol, isomalt, inositol and mannitol. In a preferred embodiment, the composition comprises a low calorie sweetener in the form of xylitol or erythritol. The inventors have found that the combination of xylitol and erythritol contributes to a pleasant experience. In particular, the inventors have found the rounded sweetness of erythritol and xylitol combined with a flavour and the formulation of the composition as powder having a finer particle size that gives a pleasant experience upon ingestion, i.e. a combination of coolness and fast melting collectively contributing to an improved mouthfeel.

The composition may comprise a combination of low calorie sweetener such as a combination of sugar alcohols a described above. Alternatively, the composition may also comprise a combination of one or more bulk sweetener and one or more intense sweeteners.

In one embodiment, the composition comprises an intense sweetener selected from the list consisting of saccharin, aspartame, stevia, sucralose and acesulfame.

The amount of low calorie sweetener may vary depending on the sweetener(s) used. In one embodiment, the low calorie sweetener is present in an amount from 5 to 95% w/w in said composition.

The composition of the present invention further comprises at least one hygroscopic agent, which functions as a moisture scavenger in order to control the water activity of the composition. In one embodiment, the at least one hygroscopic agent is selected from the list consisting of magnesium oxide, silicon dioxide, and calcium oxide.

Magnesium oxide has moisture scavenger properties, which makes it particular suitable as a hygroscopic agent in the formulation of probiotics.

The purpose of the hygroscopic agent is to ensure an optimal environment for the probiotic bacteria and thus improve the shelf life of the final product.

The inventors have further discovered that although calcium oxide have hygroscopic properties, calcium oxide has inhibitory effect on the the bacterial CFU of the composition. Since sustainably high CFU is generally an object, calcium oxide is less preferred due to the side effects on the CFUs.

While having desirable hygroscopic properties, silicon dioxide also functions as a technical excipient during the manufacturing process by contributing to the maintenance of a homogenous mixture during the preparation of the composition of the present invention. The inventors have however discovered that this desirable effect of silicon dioxide declines with elevated percentage of silicon dioxide. Thus, adding silicon dioxide (as the only hygroscopic agent) to meet the desired moisture binding capabilities will typically have an adverse impact on the manufacturing process.

The inventors have discovered that magnesium oxide does not adversely affect the manufacturing process even when present in amount significantly greater than that of silicon dioxide. Further, the inventors have discovered that magnesium oxide contributes to maintaining high CFUs in the composition.

In a preferred embodiment, the at least one hygroscopic agent is magnesium oxide. In another preferred embodiment, the composition comprises magnesium oxide and silicon dioxide, such as magnesium oxide and silicon dioxide and no further hygroscopic agent.

In a further embodiment, the composition comprises 4 to 7% w/w of magnesium oxide and optionally 0.25 to 1% w/w of silicon dioxide. In another embodiment, the composition comprises 4 to 7% w/w of magnesium oxide and 0.25 to 1% w/w of silicon dioxide. In one embodiment, In another embodiment, the composition comprises 4 to 7% w/w of magnesium oxide and 0.25 to 1% w/w of silicon dioxide and no further hygroscopic agent. In one embodiment, the composition comprises 0.25 to 1% w/w of silicon dioxide.

One disadvantage of magnesium oxide is however that it has a bitter taste that may contribute adversely to the tasting experience. The inventors have found that encapsulated magnesium oxide, for example where encapsulation material comprises a monoglyceride of an edible fatty acid, a diglyceride of an edible fatty acid, retains the moisture scavenger properties to an extend that makes it suitable for use in formulation of probiotics and reduces the potential adverse effect on the tasting experience. Accordingly, in one embodiment of the present invention the at least one hygroscopic agent is magnesium oxide and, wherein said magnesium oxide is encapsulated, for example where encapsulation material comprises a monoglyceride of an edible fatty acid, a diglyceride of an edible fatty acid, a mono/diglyceride of an edible fatty acid, a triglyceride of an edible fatty acid, a mixture of such glycerides, waxes, a propylene glycol ester of an edible fatty acid, a stearoyl lactylate, a sucrose ester of an edible fatty acid, a diacetyl tartaric acid ester of a mono- or diglyceride of an edible fatty acid, a citric acid ester of a mono- or diglyceride of an edible fatty acid and an acetic acid ester of a mono- or diglyceride of an fatty edible acid.

The amount of hygroscopic agent in the composition may vary depending on the species of hygroscopic agent employed and the moisture scavenger properties of that hygroscopic agent. In one embodiment of the present invention, the at least one hygroscopic agent is present in an amount from 0.25 to 20% w/w in said composition, such as from 0.25 to 15% w/w in said composition, for example and preferably from 0.25 to 10% w/w in said composition.

The composition of the present invention may further comprise at least one acidity regulator, which may be included to regulate and control the pH of the composition. The acidic regulator may also be contributing to the flavour of the composition. In one embodiment, the at least one acidity regulator is selected from the list consisting of citric acid, acetic acid, calcium acetate, lactic acid, malic acid, fumaric acid, tartaric acid and ascorbic acid. In another embodiment, the acidity regulator is citric acid. The amount of acidity regulator in the composition may vary. In one embodiment, the at least one acidity regulator is present in an amount from 0.25 to 5% w/w in said composition.

The composition of the present invention may further comprise an antioxidant. In one embodiment, the antioxidant is sodium ascorbate or alpha tocopherol.

The composition of the present invention comprises at least one aroma compound. More than one aroma compound may be included to provide a more complex tasting experience. In one embodiment, the at least one aroma compound is selected from the list consisting of honeydew melon aroma, blueberry aroma, peach aroma, strawberry aroma, raspberry aroma, cola aroma, chocolate aroma, peppermint aroma, cherry aroma, lemon aroma, lime aroma, orange aroma, vanilla aroma, tangerine aroma, liquorice aroma, apricot aroma, eucalyptus aroma, green tea aroma, ginger aroma and bilberry aroma. The amount of aroma compounds in the composition may vary. In one embodiment, the at least one aroma compound is present in an amount from 1 to 10% w/w in said composition.

The composition may also comprise a prebiotic that stimulates the proliferation of the microorganism in the GI of the subject ingesting the composition. In one embodiment, the composition further comprises at least one prebiotic selected from the group consisting of sialo-oligosaccharides (SOS), fructo-oligosaccharides (FOS), galacto-oligosaccharides (GOS), isomalto-oligosaccharides (IMO), xylo-oligosaccharides (XOS), arabino-xylo oligosaccharides (AXOS), mannan oligosaccharides (MOS), oligosaccharides of soy, glycosylsucrose (GS), lactosucrose (LS), sialyl-lactose (SL) Fucosyl-lactose (FL), Lacto-N-Neotetraose (LNNT), lactulose (LA), palatinose-oligosaccharides (PAO), malto-oligosaccharides, gums and/or hydrolysates thereof, pectins, starches, and/or hydrolysates thereof.

Non limiting examples of the composition of the present invention include:

A nutritional composition comprising:
(i) 0,5 to 5% w/w of at least one live microbial organism,
(ii) 5 to 95% w/w of at least one low calorie sweetener,
(iii) 0.25 to 10% w/w of at least one hygroscopic agent,
(iv) 0.25 to 5% w/w of at least one acidity regulator, and
(v) 1 to 10% w/w of at least one aroma compound.

A nutritional composition comprising:
(i) 1 to 3% w/w of at least one live microbial organism,
(ii) 70 to 95% w/w of at least one low calorie sweetener,
(iii) 0.4 to 7% w/w of at least one hygroscopic agent,
(iv) 0.25 to 1% w/w of at least one acidity regulator, and
(v) 1 to 5% w/w of at least one aroma compound.

In a preferred embodiment, the nutritional composition comprises or consists essentially of:
(i) 1 to 3% w/w of a mixture of Lactobacillus Rhamnosus GG and Bifidobacterium Lactis BL-04,
(ii) 70 to 95% w/w of a mixture of erythritol and xylitol,
(iii) 4 to 7% w/w of magnesium oxide and optionally 0.25 to 1 w/w of silicon dioxide
(iv) 0.25 to 1% w/w of citric acid, and
(v) 1 to 5% w/w of at least one aroma compound such as honeydew melon aroma.

The composition of the present invention is typically filled in a container, which is preferably sealed, which provide an oxygen and moisture barrier in order to maintain the integrity of the composition.

The water activity in the composition at the time point of filling and sealing the container is typically less than 0.15 and preferably less than 0.10, such as in the range of 0.05 to 0.10 water activity.

Accordingly, one aspect of the present invention concerns a container containing the composition of the present invention. Non limiting examples of suitable containers include a stick, bag, pouch or capsule. In a preferred embodiment, the container is an aluminium foil or a polyethylene stick, which is typically sealed by welding. The stick is typically configured for easy tear opening. The stick may have a tear notch. Preferably, the container, such as in the form of a stick, comprises a single dose of the composition. Thus, the stick may be discarded after ingestion of the composition.

In a further aspect of the present invention a kit is provided, wherein said kit comprises a plurality of containers each comprising the composition of the present invention. Preferably, each container of the kit comprises a single dose of the composition. Every container of the kit may comprise the same composition. Alternatively, the containers may comprise different compositions, e.g. the compositions may comprise different flavours, or different probiotics.

Preparation of the Composition of the Present Invention

One aspect of the present invention provides a method for preparing the composition of the present invention. The method for preparing a composition containing a microbial organism, comprises the steps of:
(a) mixing at least one microbial organism with at least one low calorie sweetener, at least one hygroscopic agent, at least one aroma compound, and optionally at least one acidity regulator and/or an antioxidant agent,
(b) reducing the water activity in the mixture obtained under step (a) to be less than 0.15, such as less than 0.10, such as in the range of 0.05 to 0.10 water activity,
(c) packaging the mixture obtained under step (b) in a container such as a sealed aluminium foil stick as described herein.

The inventors have found that higher initial colony forming units (CFU) is obtained if the composition is subjected to a drying step (step (b)) rather than subjecting the ingredients to a drying step prior to mixing the ingredients to form the composition.

In the rare event that the water activity mixture of ingredients provided in step a) do not exceed the desired water activity of the final product for example 0.15, such as less than 0.10, such as in the range of 0.05 to 0.10 water activity, step b) of reducing the water activity will not be necessary.

In a preferred embodiment, said at least one hygroscopic agent is magnesium oxide. In another preferred embodiment, said at least one hygroscopic agent comprises magnesium oxide and silicon dioxide, such as magnesium oxide and silicon dioxide and no further hygroscopic agent.

In a further embodiment, said least one hygroscopic agent comprises 4 to 7% w/w of magnesium oxide and optionally 0.25 to 1% w/w of silicon dioxide. In another embodiment, said least one hygroscopic agent comprises 4 to 7% w/w of magnesium oxide and 0.25 to 1% w/w of silicon dioxide. In one embodiment, In another embodiment, said least one hygroscopic agent comprises 4 to 7% w/w of magnesium oxide and 0.25 to 1% w/w of silicon dioxide and no further hygroscopic agent.

Most preferably, the mixture is in the form of a powder. In one embodiment, the average particle size of the powder is not larger than 200 micrometer (μm). In another embodiment, the average particle size of the powder is in the range of 45 to 200 micrometer (μm), such as 60 to 125 micrometer (μm), such as 60 to 100 micrometer, for example 75 to 90 micrometer, for example 80 micrometer. In a preferred embodiment, the average particle size of the powder is in the range of 75 to 90 micrometer (μm), for example 80 micrometer.

In one embodiment, the 35 to 95% of the particles have a size, which not larger than 200 micrometer (μm), for example at 50 to 90% of the particles have a size, which not larger than 200 micrometer (μm), such as at 60 to 80% of the particles have a size, which not larger than 200 micrometer (μm)

In one embodiment, at least 85% of the particles have a size, which falls within the range of 32 to 500 micrometer, for example at least 88% of the particles have a size in the range of 32 to 500 micrometer (μm).

A further aspect of the present invention concerns the composition obtainable from the method described above. Preferably the composition obtained by the method is a powder composition.

Uses of Nutritional Composition of the Present Invention

The composition of the present invention may be used to improve the health of a subject. The health benefit to the may be in the form of (i) improved digestion of lactose and reduce intestinal bloating, flatulence and discomfort; (ii) prevention of traveller's diarrhoea; (iii) enhancing the immune system, improving resistance to infection and improving well-being; (iv) lowering serum cholesterol levels and reducing the incidence of coronary heart disease; or (v) treating intractable diarrhoea following antibiotic therapy; (vi) reducing allergic inflammation. These health benefits may be associated with the colonization of the probiotic microorganism in the gut of the subject.

One aspect of the present invention therefore provides a method for stimulating mucin production, stimulating of the stabilization of intestinal barrier, providing antimicrobial substances to the gut, stimulating gut transit, and/or stimulating the immune system of a subject, said method comprising the steps of;
(a) administering an effective amount of the composition of the present invention to a subject in the need thereof.

Another aspect provides the composition of the present invention for stimulating mucin production, stimulating of the stabilization of intestinal barrier, providing antimicrobial substances to the gut, stimulating gut transit, and/or stimulating the immune system of a subject.

The present invention is further described in the following non-limiting items:

Item 1. A nutritional composition comprising:
(i) at least one live microbial organism,
(ii) at least one low calorie sweetener,
(iii) at least one hygroscopic agent,
(iv) at least one aroma compound,
(v) optionally, at least one acidity regulator, and
(vi) optionally, at least one antioxidant.

Item 2. The composition of item 1, wherein the composition is in the form of a powder.

Item 3. The composition according to any one of the preceding items, wherein said at least one microbial organism is a probiotic bacterium.

Item 4. The composition according to item 3, wherein said bacteria is a gram positive bacterium.

Item 5. The composition according to any one of the preceding items, wherein said at least one microbial organism is a Lactobacillales.

Item 6. The composition according to item 5, wherein said Lactobacillales is selected from the list consisting of a Lactobacillus spp., Leuconostoc spp., Pediococcus spp., Lactococcus spp., Streptococcus spp., Aerococcus spp., Carnobacterium spp., Enterococcus spp., Oenococcus spp., Sporolactobacillus spp., Tetragenococcus spp., Vagococcus spp., and Weisella spp.

Item 7. The composition according to any of the preceding items, wherein said at least one microbial organism is selected from the group consisting of Lactobacillus rhamnosus, Lactobacillus paracasei, Lactobacillus reuteri Lactobacillus acidophilus, Lactobacillus helveticus, Lactobacillus casei, Lactobacillus salivarius, Lactobacillus plantarum, Lactobacillus fermentum, Lactobacillus johnsonii, and Lactobacillus gasseri.

Item 8. The composition according to any of the preceding items, wherein said at least one microbial organism is a Lactobacillus strain selected from the group consisting of Lactobacillus rhamnosus GG (ATCC 53103), Lactobacillus rhamnosus SP1 (DSM 21690), Lactobacillus rhamnosus CGMCC 1.3724, Lactobacillus reuteri (ATCC 55730), Lactobacillus reuteri (DSM 17938) and Lactobacillus johnsonii (NCC533; CNCM 1-1225).

Item 9. The composition according to any of the preceding items, wherein said at least one microbial organism is a Lactococcus ssp.

Item 10. The composition according to any of the preceding items, wherein said at least one microbial organism is a Lactococcus ssp. selected from the group consisting of Lactococcus lactis, Lactococcus cremoris, Lactococcus diacetylactis.

Item 11. The composition according to any of the preceding items, wherein said at least one microbial organism is a Bifidobacteriales.

Item 12. The composition according to any of the preceding items, wherein said at least one microbial organism is a Bifidobacterium spp.

Item 13. The composition according to any of the preceding items, wherein the at least one microbial organism is a Bifidobacterium selected from the group consisting of Bifidobacterium lactis, Bifidobacterium longum, Bifidobacterium breve, Bifidobacterium infantis, Bifidobacterium animalis, Bifidobacterium bifidum and Bifidobacterium adolescentis.

Item 14. The composition according to any of the preceding items, wherein the one microbial organism is a Bifidobacterium strain selected from the group consisting of Bifidobacterium Lactis BI-04, Bifidobacterium lactis CNCM 1-3446 (Bb12), Bifidobacterium longum NCC3001, ATCC BAA-999 (BB536), Bifidobacterium breve Bb-03, Bifidobacterium breve M-16V, Bifidobacterium breve R0070 and Bifidobacterium infantis.

Item 15. The composition according to any one of the preceding items, wherein said at least one live microbial organism is at least one Lactobacillus spp. such as Lactobacillus Rhamnosus GG and at least one Bifidobacterium spp. such as Bifidobacterium Lactis BI-04.

Item 16. The composition according to any one of the preceding items, wherein said composition comprises Lactobacillus Rhamnosus GG and Bifidobacterium Lactis BI-04.

Item 17. The composition according to any one of the preceding items, wherein said at least one microbial organism is a probiotic fungus.

Item 18. The composition according to any of the preceding items, wherein one dose of said composition comprises 10e3 to 10e12 colony forming units of said microorganism, such as 10e6 to 10e12 colony forming units, for example 10e7 to 10e11 colony forming units, such as 10e7 to 10e11 colony forming units.

Item 19. The composition according to any one of the preceding items, wherein said composition comprises at least one low calorie sweetener selected from the list consisting of a bulk sweetener and an intense sweetener.

Item 20. The composition according to item 19, wherein said bulk sweetener is a sugar alcohol.

Item 21. The composition according to any one of the preceding items, wherein said composition comprises a bulk sweetener selected from the list consisting of xylitol, sorbitol, erythritol, maltitol, lactitol, isomalt, inositol and mannitol.

Item 22. The composition according to any of the preceding items, wherein said composition comprises an intense sweetener selected from the list consisting of saccharin, aspartame, stevia, sucralose and acesulfame.

Item 23. The composition according to any of the preceding items, wherein said at least one hygroscopic agent is selected from the list consisting of magnesium oxide, silicon dioxide, and calcium oxide.

Item 24. The composition according to any of the preceding items, wherein said at least one hygroscopic agent is magnesium oxide and silicon dioxide.

Item 25. The composition according to any of the preceding items, wherein said composition further comprises at least one acidity regulator.

Item 26. The composition according to item 25, wherein said at least one acidity regulator is selected from the list consisting of citric acid, acetic acid, calcium acetate, lactic acid, malic acid, fumaric acid, tartaric acid and ascorbic acid.

Item 27. The composition according to any of the preceding items, said at least one acidity regulator is citric acid.

Item 28. The composition according to any of the preceding items, wherein said composition further comprises at least one antioxidant.

Item 29. The composition according to any of the preceding items, wherein said antioxidant is selected from the list consisting of sodium ascorbate and alpha tocopherol.

Item 30. The composition according to any of the preceding items, wherein said at least one aroma compound is selected from the list consisting of honeydew melon aroma, blueberry aroma, peach aroma, strawberry aroma, raspberry aroma, cola aroma, chocolate aroma, peppermint aroma, cherry aroma, lemon aroma, lime aroma, orange aroma, vanilla aroma, tangerine aroma, liquorice aroma, apricot aroma, eucalyptus aroma, green tea aroma, ginger aroma and bilberry aroma.

Item 31. The composition according to any of the preceding items, wherein said at least one microbial organism is present in an amount from 0.5 to 5% w/w in said composition.

Item 32. The composition according to any of the preceding items, wherein said low calorie sweetener is present in an amount from 5 to 95% w/w in said composition.

Item 33. The composition according to any of the preceding items, wherein said at least one hygroscopic agent is present in an amount from 0.25 to 20% w/w in said composition.

Item 34. The composition according to any of the preceding items, wherein said at least one hygroscopic agent is present in an amount from 0.25 to 15% w/w in said composition.

Item 35. The composition according to any of the preceding items, wherein said at least one hygroscopic agent is present in an amount from 0.25 to 10% w/w in said composition.

Item 36. The composition according to any of the preceding items, wherein said at least one aroma compound is present in an amount from 1 to 10% w/w in said composition.

Item 37. The composition according to any of the preceding items, wherein said at least one acidity regulator is present in an amount from 0.25 to 5% w/w in said composition.

Item 38. The composition according to any of the preceding items, wherein said composition comprises
(i) 0,5 to 5% w/w of at least one live microbial organism,
(ii) 5 to 95% w/w of at least one low calorie sweetener,
(iii) 0.25 to 10% w/w of at least one hygroscopic agent,
(iv) 0.25 to 5% w/w of at least one acidity regulator, and
(v) 1 to 10% w/w of at least one aroma compound.

Item 39. The composition according to any of the preceding items, wherein said composition comprises
(i) 1 to 3% w/w of at least one live microbial organism,
(ii) 70 to 95% w/w of at least one low calorie sweetener,
(iii) 0,4 to 7% w/w of at least one hygroscopic agent,
(iv) 0.25 to 1% w/w of at least one acidity regulator, and
(v) 1 to 5% w/w of at least one aroma compound.

Item 40. The composition according to any of the preceding items, wherein said composition comprises
(i) 1 to 3% w/w of a mixture of Lactobacillus Rhamnosus GG and Bifidobacterium Lactis BL-04,
(ii) 70 to 95% w/w of a mixture of erythritol and xylitol,
(iii) 4 to 7% w/w of magnesium oxide and optionally 0.25 to 1% w/w of silicon dioxide
(iv) 0.25 to 1% w/w of citric acid, and
(v) 1 to 5% w/w of at least one aroma compound such as honeydew melon aroma.

Item 41. The composition according to any of the preceding items further comprising at least one prebiotic.

Item 42. The composition according to any of the preceding items further comprising at least one prebiotic selected from the group consisting of sialo-oligosaccharides (SOS), fructo-oligosaccharides (FOS), galacto-oligosaccharides (GOS), isomalto-oligosaccharides (IMO), xylo-oligosaccharides (XOS), arabino-xylo oligosaccharides (AXOS), mannan oligosaccharides (MOS), oligosaccharides of soy, glycosylsucrose (GS), lactosucrose (LS), sialyl-lactose (SL) Fucosyl-lactose (FL), Lacto-N-Neotetraose (LNNT), lactulose (LA), palatinose-oligosaccharides (PAO), malto-oligosaccharides, gums and/or hydrolysates thereof, pectins, starches, and/or hydrolysates thereof.

Item 43. The composition according to any of the preceding items, wherein the particle size of the powder is in the range of 60 to 125 μm (micrometer), such as 60 to 100 μm, for example 75 to 90 μm, for example 80 μm.

Item 44. The composition according to any of the preceding items, wherein said at least one hygroscopic agent is magnesium oxide and, wherein said magnesium oxide is encapsulated, for example where encapsulation material comprises a monoglyceride of an edible fatty acid, a diglyceride of an edible fatty acid, a mono/diglyceride of an edible fatty acid, a triglyceride of an edible fatty acid, a mixture of such glycerides, waxes, a propylene glycol ester of an edible fatty acid, a stearoyl lactylate, a sucrose ester of an edible fatty acid, a diacetyl tartaric acid ester of a mono- or diglyceride of an edible fatty acid, a citric acid ester of a mono- or diglyceride of an edible fatty acid and an acetic acid ester of a mono- or diglyceride of an fatty edible acid.

Item 45. The composition according to any of the preceding items, wherein the water activity in said composition is less than 15%, such as less than 10%, such as in the range of 5% to 10% water activity.

Item 46. The composition according to any of the preceding items, wherein said composition comprises 4 to 8% w/w of said at least one hygroscopic agent.

Item 47. The composition according to any of the preceding items, wherein said at least one hygroscopic agent is magnesium oxide and silicon dioxide.

Item 48. The composition according to any of the preceding items, wherein said composition comprises 4 to 7% w/w of magnesium oxide and optionally 0.25 to 1% w/w of silicon dioxide.

Item 49. The composition according to any of the preceding items, wherein said composition comprises 0.25 to 1% w/w of silicon dioxide.

Item 50. The composition according to any of the preceding items, wherein 35 to 95% of the particles of the powder composition have a particle size not larger than 200 micrometer (μm), for example at 50 to 90% of the particles have a size not larger than 200 micrometer (μm), such as at 60 to 80% of the particles have a size not larger than 200 micrometer (μm).

Item 51. A method for preparing a composition containing a microbial organism, said method comprising the steps of:
(a) mixing at least one microbial organism with at least one low calorie sweetener, at least one hygroscopic agent, at least one aroma compound, and optionally at least one acidity regulator and/or an antioxidant agent,
(b) reducing the water activity in the mixture obtained under step (a) to be less than 15%, such as less than 10%, such as in the range of 5% to 10% water activity,
(c) packaging the mixture obtained under step (b) in a container.

Item 52. The method for preparing a composition containing a microbial organism according to item 51, wherein said composition is a powder composition.

Item 53. The method according to any of the preceding items, wherein said composition comprises 4 to 8% w/w of said at least one hygroscopic agent.

Item 54. The method according to any of the preceding items, wherein said at least one hygroscopic agent is magnesium oxide and silicon dioxide.

Item 55. The method according to any of the preceding items, wherein said composition comprises 4 to 7% w/w of magnesium oxide and optionally 0.25 to 1% w/w of silicon dioxide.

Item 56. The method according to any of the preceding items, wherein said composition comprises 0.25 to 1% w/w of silicon dioxide.

Item 57. The method according to any of the preceding items, wherein 35 to 95% of the particles of the powder composition have a particle size not larger than 200 micrometer (μm), for example at 50 to 90% of the particles have a size not larger than 200 micrometer (μm), such as at 60 to 80% of the particles have a size not larger than 200 micrometer (μm).

Item 58. The composition obtainable from the method according to any of the preceding items.

Item 59. A container containing the composition according to any one of the preceding items.

Item 60. The container according to item 59, wherein said container is selected from the list consisting of a stick, bag, pouch or capsule.

Item 61. The container according to item 59 or 60 comprising one dose of said composition.

Item 62. A kit comprising a plurality of containers according to any one of items 59 to 61.

Item 63. A method for stimulating mucin production, stimulating of the stabilization of intestinal barrier, providing antimicrobial substances to the gut, stimulating gut transit, and/or stimulating the immune system of a subject, said method comprising the steps of;
(a) administering an effective amount of the composition according to any one of the preceding items to a subject in the need thereof.

Item 64. The composition according to any one of the preceding items for stimulating mucin production, stimulating of the stabilization of intestinal barrier, providing antimicrobial substances to the gut, stimulating gut transit, and/or stimulating the immune system of a subject.

When describing the embodiments (or items) of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The term “comprising”, “comprise” and “comprises” herein are intended by the inventors to be optionally substitutable with the terms “consisting of”, “consist of” and “consists of”, respectively, for every embodiment disclosed herein.

### EXAMPLES

### Example 1

The method for preparing the composition of the present invention containing a microbial organism generally comprises the seteps, said method comprising the steps of:
Weighing off of raw materials according to a master recipe.
Mixing. The raw materials are mixed together in specific order to ensure homogeneity of the finished blend. Mixing time and sequence is specified to minimize the loss of viable probiotic cells during the process.
Storage. Water activity is measured after mixing using a Rotronic hygrolab at ambient temperature. 2-3 grams of powder is placed in test vehicle, the sensor is placed directly above the samples, sealing the sample chamber from ambient conditions. Water activity is measured when equilibrium is achieved.
Filling. Water activity is measured after storage. The powder is filled in a container, preferably into aluminium sticks. Welding integrity is controlled routinely as well as the opening mechanism and batch print.
Release. Microbiological analysis is performed to secure concentration of probiotic bacteria is according to release specification as well as no impurities exceed the control limits. This is done by standard plating of a dissolved, diluted, sample of the product, following incubation on growth media and counting of grown colonies.

In one example, the composition of the present invention is prepared by
Weighing off of raw materials according to a master recipe. Inactive raw materials as well as probiotic are release according to internal specifications including moisture content. Probiotic strains are kept in freezer in sealed aluminium bags prior to use, and are acclimatized to the temperature of the production area, before opened.
Mixing. The raw materials are mixed together in specific order to ensure homogeneity of the finished blend. Mixing time and sequence is specified to minimize the loss of viable probiotic cells during the process. Following sequence and times are according to a 500 kg batch mixed in appropriate sized double cone mixer.
Mixing step 1. Lactobacillus Rhamnosus GG and Bifidobacterium Lactis are mixed with parts of the Erythritol. Mix for 10 minutes.
Sieving step 1. Magnesium oxide, Silicon dioxide and parts of the Xylitol are sieved through mesh size 1.2 mm.
Mixing step 2. Sieving step 1 is mixed with Rest of the xylitol, Aroma, citric acid. Mix briefly.
Mixing step 3. Mixing step 1 and 2 is combined and mixed. Mix for 10 minutes.
Mixing step 4. Rest of the Erythritol is added to mixing step 3. Mix for 15 minutes.

Water activity is measured after mixing
Drying. Regardless of moisture content, the blend is stored with desiccant bags approved for contact with food products, until acceptable water activity is obtained. Water activity is measured after drying, and the blend is released for next process step if values are below limits.
Filling. Filling of powder into aluminium sticks is carried out under controlled atmosphere with a relative humidity not higher than 25%. Welding integrity is controlled routinely as well as the opening mechanism and batch print.
Release. Microbiological analysis is performed to secure concentration of probiotic bacteria is according to release specification as well as no impurities exceed the control limits. Water activity is measured and product released only if values are below limits.

Selection of Bulk/Low Calorie Sweetener

The inventors have tested a wide range of compositions including various ingredients such as various sweeteners and on based the test results Erythritol and Xylitol are preferred sweeteners.

Both the materials contribution to the desired mouthfeel. The composition comprising Erythritol and Xylitol, compared to the other candidates, showed to be quickly dissolving, as well as having a cooling, fresh feel. Further, the composition did not appear to stay in the mouth or feel dry as many of the other tested materials did. Besides the above technical properties, both Erythritol and xylotol are almost free of calories and were non-cariogenic.

Determining the Best Particle Size for the Formulation

The ratio between small and larger particles is very specific to the choice of low calorie sweetener. A tasting panel indicated that a finer material was preferred as it gave a better experience and a faster melt upon ingestion. This, however, needs to be counter-balanced with a fraction of coarser material in order to make the final product more free flowing to ensure good production and minimizing dust formation. The inventors found that the mouth-feel is very much affected by the ratio of fine and coarser powders, making it necessary to balance both the cooling effect, which is more pronounced in some materials such as xylitol and the finer the powder is, and the melting time, which is prolonged with coarser material.

Tasting Evaluation

The tasting panel was instructed to ingest 1 gram of the composition by pouring the composition from the stick directly unto the tongue. The tasting panel reported an instant cooling effect. Upon contact with moisture (in this case saliva), the composition dissolves very quickly. The high negative heat of solution, a characteristic for Erythritol and Xylitol, means that dissolving the compounds requires energy instead of releasing it, which in turn is experienced as a cooling effect. The rounded sweetness of Erythritol and Xylitol combined with a flavor results in a pleasant experience with no disagreeable aftertaste or lingering effect.

Product Stability—Low Moisture Content.

Moisture content and water activity only being some of the criteria for selecting raw materials, the inventors found that it was necessary to introduce a drying step in the formulation process. This was found not possible to reach desired water activity levels on raw material level without adversely affecting the stability of the composition. Leading us to investigate several combinations in regards of raw material quality and particle size. It was finally determined that an acceptable level for water activity could be achieved on the final blend. In conclusion it is paramount for the product quality to have a drying step at a specific place in the sequence of process steps.

### Example 2

The below table includes the data obtained by determining the particle size distribution in a sample a

| | | |
|---|---|---|
| ≥1000 | μm | 0.12% |
| 500-1000 | μm | 3.12% |
| 250-500 | μm | 18.05% |
| 125-250 | μm | 24.91% |
| 63-125 | μm | 21.60% |
| 32-63 | μm | 24.30% |
| ≤32 | μm | 7.92% |

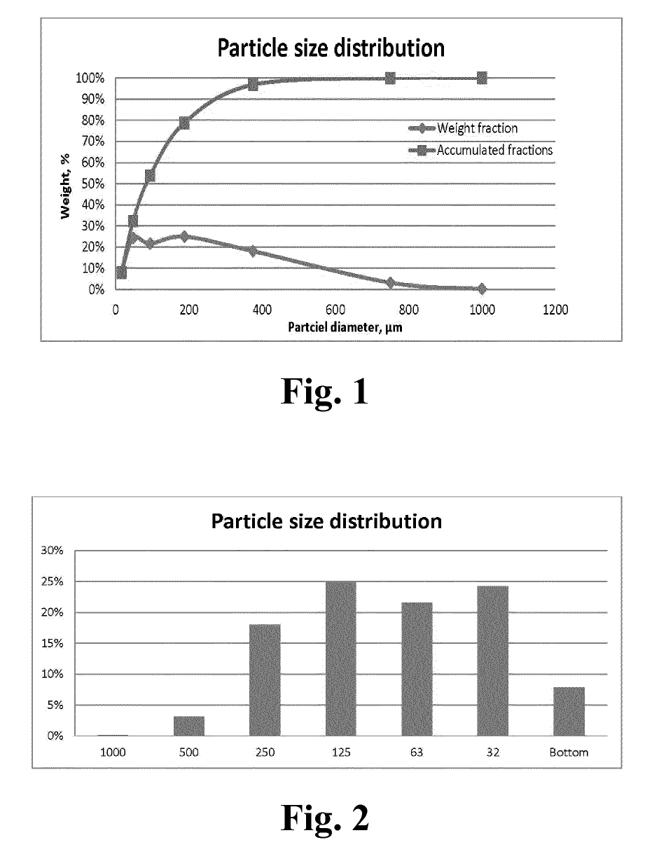

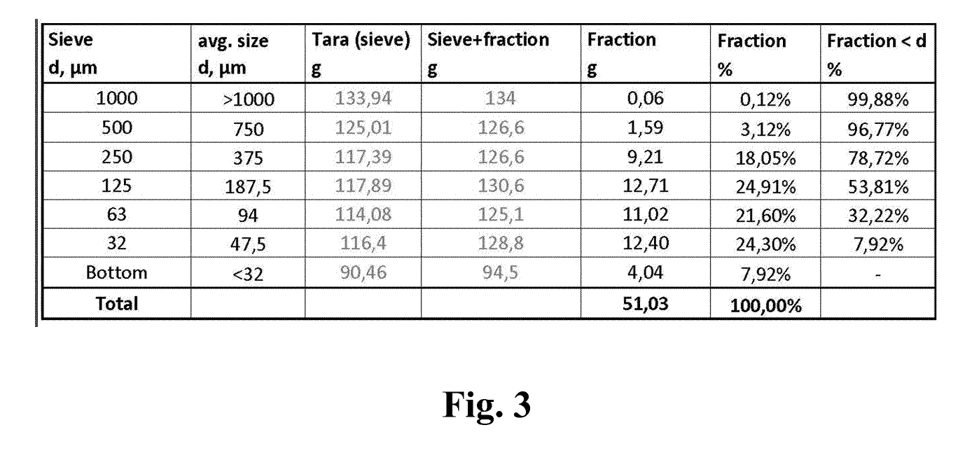

Further data on the particle size distribution is disclosed in FIGS. 1 to 3.

1. A nutritional powder composition comprising:
at least one live probiotic bacterium,
(ii) at least one low calorie sweetener,
(iii) at least one hygroscopic agent, and
(iv) at least one aroma compound,
(v) optionally, further comprising at least one acidity regulator, and
(vi) optionally, further comprising at least one antioxidant.

2-18. (canceled)

19. The composition according to claim 1, wherein said at least one hygroscopic agent is selected from the group consisting of magnesium oxide, silicon dioxide, and calcium oxide.

20. The composition according to claim 1, wherein said at least one hygroscopic agent is magnesium oxide.

21. The composition according to claim 1, wherein said composition comprises 4 to 8% w/w of said at least one hygroscopic agent.

22. The composition according to claim 1, wherein said at least one hygroscopic agent is magnesium oxide and silicon dioxide.

23. The composition according to claim 1, wherein said composition comprises 4 to 7% w/w of magnesium oxide and optionally 0.25 to 1% w/w of silicon dioxide.

24. The composition according to claim 1, wherein 35 to 95% of the particles of the powder composition have a particle size not larger than 200 micrometer.

25. The composition according to claim 1, wherein said composition comprises at least one low calorie sweetener in the form of a bulk sweetener selected from the group consisting of xylitol, erythritol, sorbitol, maltitol, lactitol, isomalt, inositol and mannitol.

26. The composition according to claim 1, wherein said at least one probiotic bacterium is a Lactobacillales selected from the group consisting of a Lactobacillus spp., Leuconostoc spp., Pediococcus spp., Lactococcus spp., Streptococcus spp., Aerococcus spp., Carnobacterium spp., Enterococcus spp., Oenococcus spp., Sporolactobacillus spp., Tetragenococcus spp., Vagococcus spp., and Weisella spp.

27. The composition according to claim 1, wherein said at least one probiotic bacterium is selected from the group consisting of Lactobacillus rhamnosus, Lactobacillus paracasei, Lactobacillus reuteri, Lactobacillus acidophilus, Lactobacillus helveticus, Lactobacillus casei, Lactobacillus salivarius, Lactobacillus plantarum, Lactobacillus fermentum, Lactobacillus johnsonii, Lactobacillus gasseri, Lactobacillus rhamnosus GG (ATCC 53103), Lactobacillus rhamnosus SP1 (DSM 21690), Lactobacillus rhamnosus CGMCC 1.3724, Lactobacillus reuteri (ATCC 55730), Lactobacillus reuteri (DSM 17938) and Lactobacillus johnsonii (NCC533; CNCM I-1225).

28. The composition according to claim 1, wherein said at least one probiotic bacterium is a Lactococcus ssp. selected from the group consisting of Lactococcus lactis, Lactococcus cremoris, and Lactococcus diacetylactis.

29. The composition according to claim 1, wherein said at least one probiotic bacterium is a Bifidobacterium spp. selected from the group consisting of Bifidobacterium lactis, Bifidobacterium longum, Bifidobacterium breve, Bifidobacterium infantis, Bifidobacterium animalis, Bifidobacterium bifidum and Bifidobacterium adolescentis, Bifidobacterium Lactis BI-04, Bifidobacterium lactis CNCM 1-3446 (Bb12), Bifidobacterium longum NCC3001, ATCC BAA-999 (BB536), Bifidobacterium breve Bb-03, Bifidobacterium breve M-16V, Bifidobacterium breve R0070 and Bifidobacterium infantis.

30. The composition according to claim 1, wherein one dose of said composition comprises 10³ to 10¹² colony forming units of said microorganism.

31. The composition according to claim 1, wherein said at least one hygroscopic agent is present in an amount from 0.25 to 20% w/w in said composition.

32. The composition according claim 1, wherein said composition comprises:
(i) 1 to 3% w/w of a mixture of Lactobacillus Rhamnosus GG and Bifidobacterium Lactis BL-04,
(ii) 70 to 95% w/w of a mixture of erythritol and xylitol,
(iii) 4 to 7% w/w of magnesium oxide and optionally 0.25 to 1 w/w of silicon dioxide
(iv) 0.25 to 1 w/w of citric acid, and
(v) 1 to 5 w/w of at least one aroma compound such as honeydew melon aroma.

33. The composition according to claim 1, wherein the water activity in said composition is in the range of 5% to 10% water activity.

34. A method for preparing a powder composition containing a probiotic bacterium, said method comprising:
(a) mixing at least one probiotic bacterium with at least one calorie sweetener, at least one hygroscopic agent, at least one aroma compound, and optionally at least one acidity regulator or an antioxidant agent,
(b) where the water activity in the mixture obtained under step (a) exceeds 15%, reducing the water activity in the mixture to be in the range of 5% to 10% water activity, and
(c) packaging the mixture obtained under step (b) in a container.

35. The powder composition obtainable from the method according to claim 34.
